(19) United States
(12) Patent Application Publication
(10) Pub. No.: US 2022/0275430 A1
(43) Pub. Date: Sep. 1, 2022

(54) METHOD FOR DETECTING AND QUANTIFYING A BIOLOGICAL SPECIES OF INTEREST BY METAGENOMIC ANALYSIS, TAKING INTO ACCOUNT A CALIBRATOR

(71) Applicant: BIOMERIEUX, Marcy l'Etoile (FR)

(72) Inventors: Vladimir Lazarevic, Renens (CH); Sébastien Hauser, Voreppe (FR); Maud Tournoud, Sassenage (FR)

(73) Assignee: BIOMERIEUX, Marcy l'Etoile (FR)

(21) Appl. No.: 17/629,065

(22) PCT Filed: Jul. 22, 2020
(86) PCT No.: PCT/EP2020/070716

§ 371 Date: Jan. 21, 2022

(30) Foreign Application Priority Data
Jul. 23, 2019 (FR) FR1908366

(51) Int. Cl. C12Q 1/689

(52) U.S. Cl. CPC C12Q 1/689; C12Q 1/6869

(57) ABSTRACT

A method for detecting a biological species of interest (SOI) potentially present in an analysis sample, the biological species of interest having a known or partially known genome, the analysis sample comprising a mixture of various biological species, the method
comprising (a) extracting nucleic acids from the analysis sample; (b) sequencing nucleotide sequences extracted, (c) on the basis of the sequencing result, (i) assigning the sequences resulting from the sequencing, based on a reference database of sequences; (i) assigning sequences; (ii) determining a quantity (R_{SOI}, RN_{SOI}) of sequences assigned to the biological species of interest; and, prior to the sequencing, adding a calibrator, the calibrator being a biological species added in a known concentration, to the analysis sample, the calibrator having a known genome, and on the basis of the sequencing result, determining a quantity (R_{CAL}) of sequences assigned to the calibrator; (d) on the basis of the quantities of sequences estimated in the determining of (RSOI, RNSOI) and (R_{CAL}), estimating a concentration (C_{SOI}) of the biological species of interest (SOI) in the sample.

### TECHNICAL FIELD

The technical field of the invention is the identification of a biological species of interest by metagenomic analysis.

### PRIOR ART

Amplification of nucleic acids by polymerase chain reaction (PCR) allows a rapid and early diagnosis to be made as regards the presence of certain microorganisms in a sample. PCR is for example particularly suitable for detecting the deoxyribonucleic acid (DNA) of bacteria that are difficult to cultivate, or that develop slowly, such as Mycobacterium tuberculosis.

However, implementation of PCR requires the use of primers, which specifically target a gene present in a target biological species. Thus, PCR allows an analysis specific to one biological species, this making it a sensitive, selective method that may be quantitative. However, it assumes prior knowledge regarding the targeted biological species. If a plurality of biological species are sought, so-called multiplex PCRs must be carried out, this making the process more complex.

It is also possible to target a gene present in various target biological species. As regards bacteria, it may for example be a question of the 16S RNA gene. The PCR analysis is then said to be broad-range. However, broad-range PCR is trickier to implement, and assumes prior knowledge regarding the target biological species to be identified is available. Targeting a gene is described in EP2985350 or in the publication by Stämmler F. “Adjusting microbiome profiles for differences in microbial load by spike-in bacteria”, Microbiome (2016) 4, 28.

In contrast to the techniques described above, metagenomics allows the genomes of a plurality of individuals of different biological species in a given medium to be sequenced. It is then possible to determine the species actually present in the sample, and their relative abundances. Metagenomics sequences the genomes of a plurality of individuals of different species in a given medium, and does so without prior knowledge regarding the biological species in the sample, whether they be bacterial, viral or human. An analysis of the various genomes of the biological species in a sample is thus obtained. It is then possible to determine which species are present, and their relative abundances.

Progress has recently been made in the field of sequencing, with the advent of the second- and third-generation sequencing technologies designated HTS technologies, HTS standing for high-throughput sequencing. The performance of bioinformatics, which allows rapid computational processing of the biological information generated by sequencing, has improved. At the present time, high-throughput sequencing allows enough sequences to be generated to obtain a representative inventory of the various species present in the sample. It is a commercially available analyzing method, use of which has become relatively common. Document WO2018/069430 describes an application of a metagenomic analysis to identification of pathogenic agents and markers of resistance to antibiotics.

The publication by Ruppé E “Clinical metagenomics of bone and joint infections: a proof of concept study”, also describes the application of metagenomics to identification of bacteria. Document WO2017/053446 and the publication by Schlaberg “Validation of metagenomic next-generation sequencing tests for universal pathogen detection” describe metagenomic methods for analyzing samples, in which an internal control, formed by a known biological species, is introduced into the sample.

The inventor provides a method for detecting, and potentially quantifying, a biological species of interest, or even various biological species of interest, in a sample, by carrying out a metagenomic analysis of the sample. In addition, the method allows an indicator as to whether the biological or bioinformatical steps of the metagenomic process are progressing correctly to be established.

### SUMMARY OF THE INVENTION

One subject of the invention is a method for detecting a biological species of interest potentially present in an analysis sample, the biological species of interest having a known or partially known genome, the analysis sample comprising a mixture of various biological species, the method comprising the following steps:
a) extracting nucleic acids from the analysis sample;
b) sequencing the nucleotide sequences extracted in step a);
c) on the basis of the result of the sequencing:
   (i) assigning the sequences resulting from step b), based on a reference database of sequences;
   (ii) determining a quantity of sequences assigned to the biological species of interest;
the method being characterized in that it comprises, prior to step b), adding a calibrator, the calibrator being a biological species added in a known concentration, to the analysis sample, the calibrator having a known genome, and in that step c) comprises
   (iii) determining a quantity of sequences assigned to the calibrator;
d) on the basis of the quantities of sequences estimated in steps (ii) and (iii), estimating a concentration of the biological species of interest in the sample.

Preferably, in sub-steps ii) and iii), the quantities of sequences respectively assigned to the biological species of interest and to the control biological species are normalized by a reference quantity. The reference quantity may for example be a total quantity of sequences produced during the sequencing.

The method may comprise taking into account a decision threshold, to which the concentration of the species of interest is intended to be compared.

The decision threshold is preferably expressed in units corresponding to a number of sequences per unit volume (or per unit weight), and for example in genome equivalent per mL. The decision threshold may depend on the biological species in question.

Preferably, the calibrator has one of the characteristics described below, implemented in isolation or in technically achievable combinations:
the calibrator is such that the size of its genome is comprised between 0.1 times to 10 times the size of the genome of the biological species of interest;
the sample comprising endogenous organisms, the calibrator has a genome different from that of the endogenous organisms;
the concentration of the calibrator is comprised between 0.001 times and 1000 times, and preferably between 0.01 and 100 times the decision threshold taken into account;
the biological species of interest is a bacterium, the calibrator having an intact membrane or cell wall;
the biological species of interest is a virus, the calibrator having a protein shell;
the genome of the calibrator has a number of GC (guanine—cytosine) bases comprised between 75% and 125% of the number of GC (guanine—cytosine) bases of the genome of the biological species of interest.

### Step d) May Comprise:

determining a first ratio, between the quantities of sequences respectively assigned to the biological species of interest and to the calibrator;
determining a second ratio, between the respective genome sizes of the calibrator and of the biological species of interest;
taking into account the calibrator concentration added to the analysis sample.

Estimating the concentration of biological species of interest may then comprise computing a product of the first ratio multiplied by the second ratio and by the concentration of the calibrator added to the analysis sample.

### Step d) May Comprise:

determining a coverage for the biological species of interest and for the calibrator;
computing a ratio between the coverage determined for the biological species of interest and the coverage determined for the calibrator;
multiplying the ratio thus computed by the calibrator concentration added to the sample.

The method may comprise, following step d), a step e) of taking into account the decision threshold and of comparing the concentration resulting from step d) with the decision threshold.

Other advantages and features will become more clearly apparent from the following description of particular embodiments of the invention, which are provided by way of nonlimiting examples, and which are shown in the figures listed below.

### FIGURES

FIG. 1 schematically shows the main steps of a method according to the invention.

FIG. 2A shows a comparison of quantifications of a biological species of interest, in fact S.aureus, respectively obtained by implementing the steps described below (y-axis) and a reference method (x-axis) employing culture.

FIG. 2B shows a comparison of quantifications of a biological species of interest, in fact S.aureus, respectively obtained by implementing the steps described below (y-axis) and a reference method (x-axis) employing quantitative PCR.

FIG. 3 shows a statistical distribution of the normalized quantity of sequences, corresponding respectively to various biological species of interest, measured on test samples considered not to comprise said biological species of interest.

FIG. 4 is a figure showing a comparison between concentrations of biological species of interest respectively estimated by culture (x-axis) and by metagenomic analysis (y-axis).

### DESCRIPTION OF PARTICULAR EMBODIMENTS

The objective of the method is to be able to detect the presence of a biological species of interest SOI in a sample. In case of detection, the method may allow an absolute quantification of the species of interest SOI, so as to allow a comparison with a decision threshold SD.

By biological species, what is meant is a microorganism, for example a bacterium, or a virus, a fungus, an archaebacterium, an amoeba, a protist, or a microalgae. A biological species may also be a cell or any other thing or entity comprising a sequence for nucleic acid.

When the sample is obtained from a human or animal organism, the biological species of interest may be a pathogenic species. When the sample is obtained by sampling from an industrial process or from the environment, the biological species of interest may be a species considered to be a contaminant, or a species of interest having an importance in an industrial process or in the environment, and the presence or concentration of which it is desired to ascertain.

The species of interest has a known, or partially known, genome. The genome, or its known segment, is made up of sequences, which are referred to as sequences of interest.

The method may address a plurality of species of interest simultaneously. Thus, the term a species of interest is to be interpreted as meaning at least one species of interest.

The decision threshold SD is a threshold that it makes it possible to characterize a load of the biological species of interest, of a microorganism for example, depending on the targeted application. It is for example set in light of a regulatory, or sanitary or industrial limit. For example, when the application is used in assistance with clinical diagnosis, the biological species of interest being a bacterium, the decision threshold may be a concentration below which the presence of the bacterium corresponds to a colonization, i.e. a non-pathological development, and above which the presence of the bacterium is considered to be pathological, and for example to correspond to an infection. When the invention is applied to an industrial process, the detection threshold corresponds to a pass value, such that above the detection threshold the sample is considered not to pass, and below the detection threshold the sample is considered to pass. Whatever the application, when the concentration of the biological species of interest is higher than or equal to the decision threshold, it is defined as being critical. In certain applications, for example in the manufacture of fermented products, a concentration of biological species of interest may be considered to be critical if it is lower than a decision threshold, the latter corresponding to a minimum acceptable concentration of the biological species.

The sample is generally a sample that will have been sampled from the environment or from a dead or living organism, or even from a manufactured product or a product associated with food production. The sample may also have been sampled from an industrial facility, for the sake of process control. Thus, the sample comprises various biological species, not having the same genome. In particular, when the sample results from sampling of an organism, for example a human or animal organism, the sample comprises a significant quantity of cells originating from the sample organism, these cells possibly even making up most of the sample. The genomes of human or animal organisms have a size that is 1000 to 100 000 times larger than the genomes of prokaryotic organisms. In addition, the sample generally comprises biological species that are naturally present in the sample, and not liable to result in a pathology or a critical contamination. For example, when the sample is a bronchoalveolar sample, it comprises a bacterial flora naturally present in the lungs. When the sample is a stool sample, it comprises a bacterial flora naturally present in the digestive tract. Hence, when the biological species of interest is a bacterium or a virus, the nucleic acids of the biological species of interest may be a minority of the nucleic acids in the sample.

The sample comprises what may be referred to as “matrix” species, which are endogenous to the sample, and which are liable to mask metagenomic information relative to the biological species of interest. For example, when the sample is taken from a yoghurt, from a piece of meat or from a vaccine, it comprises matrix species that are representative of these media. In the case of a sample taken from an organism, the matrix comprises constituent cells of the organism.

One important aspect of the invention is that the sample undergoes extraction of nucleic acids (DNA and/or RNA), followed by a sequencing process, according to the principles of metagenomic analysis. The sequencing process may be preceded by an amplifying process. The sequencing may be whole-genome sequencing (WGS), and notably whole-genome shotgun sequencing. An inventory of sequences of genes of the various species of the sample is thus obtained. All, or almost all, of the nucleic acid of the various species of the sample is sequenced, using a high-throughput sequencing method. Bioinformatical means then allow sequences of interest, associated with the biological species of interest, to be identified and a quantity thereof, generally a normalized quantity thereof, to be determined as described below. The bioinformatical means are based on a database of reference sequences, for example of complete reference genomes in the context of a WGS process such as mentioned above. The database comprises at least the, whole or partial, genomes of the biological species of interest that are potentially present in the sample. It also comprises the, whole or partial, genome of a biological species referred to as the control species, the latter being described below.

Thus, with this technique, by sequencing, a genomic description of the various species of the sample is obtained. Next, among the inventoried genomic sequences, the sequences corresponding to the biological species of interest and those corresponding to the control species are identified.

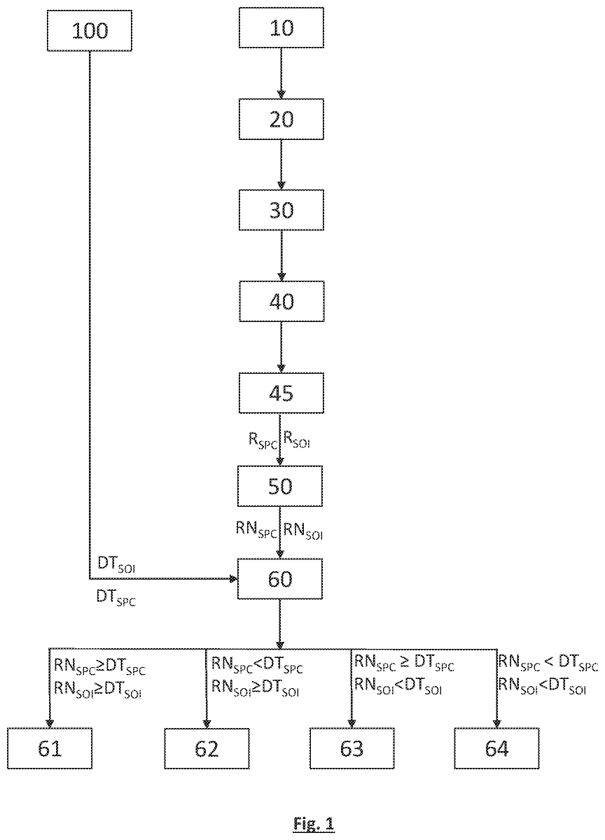

The method comprises the steps described below, with reference to FIG. 1.

### Step 10: Taking the Sample.

In this example, the sample is taken from a living human organism, for the sake of assisting with diagnosis. However, the invention is not limited to an application to the realm of living things. The sample may be taken from an industrial or hospital environment, so as to verify a conformity with respect to a decision threshold.

### Step 20: Adding a Control Species.

One of the objectives of the invention is to evaluate to what extent a metagenomic analysis is exploitable. It is in particular a question of evaluating a conformity of all of the steps from preparation of the sample, sampling excluded, to bioinformational analysis of the sequencing data. To this end, a control species, denoted SPC, acronym of sample processing control, is added to the sample. One function of the control species is to allow whether the steps of extracting nucleic acids and of sequencing, which steps are described below, are progressing correctly to be checked. The control species SPC may be a known biological species, the genome of which is also known, preferably in its entirety. The control species SPC may be a natural biological species. It may also be an artificial species, for example an encapsidated RNA (ribonucleic acid). Preferably, the control species SPC is not initially present in the sample, or if so in a negligible quantity. Preferably, the content of control species SPC initially present in the sample, i.e. present before the addition, is preferably at least 10 times lower, or preferably at least 100 or 1000 times lower, than the concentration CSPC of the control species SPC added to the sample. The control species SPC may for example be a bacterium. It is important for the concentration of the control species added to be controlled.

The control species may be chosen taking into account the aspects listed below:
a) The control species must preferably differ from the organisms naturally present in the sample, or endogenous organisms, and from the sought-after species of interest: thus, the bioinformational tool will be able to accurately identify sequences generated by sequencing the SPC.
b) The quantity of sequences assigned to the control species, during sequencing, must be sufficient to be able to be detected correctly, without however masking the useful information, corresponding to the sequences of the biological species of interest. In other words, the control species is preferably detectable by high-throughput sequencing, while not being preponderant in the sample. In particular, when it is desired to determine a positiveness (concentration of the species above the decision threshold) or a negativeness (concentration of the species below the decision threshold), it is preferable for the control species to be such that:
   The size of its genome is preferably similar, or at least comparable, to the size of the genome of the biological species of interest. More particularly, the size of the genome of the control species is comprised between 0.1 times to 10 times the size of the genome of the biological species of interest.
   The concentration CSPC of the control species may be set depending on the decision threshold. The concentration CSPC of the control species SPC added may for example be comprised between 0.001 times and 1000 times, and preferably between 0.01 and 100 times, the decision threshold.
   The nucleic acids of the control species SPC undergo a similar treatment to the nucleic acids of the species of interest in the steps of preparing the sample, of extracting and of sequencing, and preferably:
      the percentage of GC (guanine, cytosine) bases is preferably close to the percentage of GC bases of the biological species of interest; by close to, what is meant is comprised between 75% and 125%, and preferably between 80% and 120%.
      The control biological species preferably comprises, when the biological species of interest is a bacterium, an intact cell wall or a membrane, or, when the biological species of interest is a virus, a protein shell. This condition furthermore allows the steps of lysing or of extracting nucleic acids of the biological species of interest to be monitored.
c) Preferably, the nucleotide sequences of the control species do not contain genomic markers, such as for example markers of resistance to antibiotics, or virulence markers, so as not to cause the results of a potential test of sensitivity to antibiotics to be corrupted by the presence of such markers in the genome of the biological species of interest. Preferably, the nucleotide sequences of the control species do not contain any other gene of clinical or industrial interest and the presence of which is liable to be checked for.
d) The control species is preferably easily manipulatable, and in particular:
   harmless to humans or to the environment;
   and/or resistant to heat treatments such as freeze-drying or freezing, this facilitating storage.
e) The control species must not form spores, or if so only marginally.
f) The control species must have a sensitivity to lysis close to that of the biological species of interest.
g) The control species is available in the form of balls, each ball comprising a calibrated concentration of control biological species in freeze-dried form.

It will be noted that a single control species SPC may be used, or that a plurality of control species, of various types, may be used. Various control biological species may be used for a given biological species of interest. According to one possibility, the control species forms a calibrator. According to another variant, a calibrator, different from the control species, is added to the sample. The calibrator allows the concentration of the species of interest to be estimated. This alternative, which corresponds to a variant of the invention, is described after the description of steps 61 to 64. See the section titled “Variant”.

The added concentration CSPC of the control species SPC is preferably known with precision. Specifically, it may allow, provided that certain conditions are met, the concentration of biological species of interest in the sample to be quantified, the control species then forming a calibrator. The term added concentration designates the concentration of the control species in the sample due to the addition of the control species.

In the description of steps 30 to 60, the addition of a single type of control species to the sample is described, by way of advantageous example. The control species then performs the function of quality control in the steps of the metagenomic analysis, and the function of calibrator, allowing a quantification of the concentration of the biological species of interest.

At the end of step 20, a concentration CSPC of the control species will have been added to the sample. The added concentration CSPC may be expressed in GEq/mL (genome equivalent per mL).

### Step 30: Lysing and Extracting Nucleic Acids.

In this step, the cells of the sample, and notably the cells of the biological species of interest and of the control species, undergo a lysis, in order to allow their DNA to be extracted. Various strategies may be envisioned:
The lysis may be parameterized to preferentially target the biological species of interest;
The control species must have the same sensitivity to lysis as the biological species of interest, or a sensitivity to lysis that may be considered equivalent.
The lysis may include a first lysis, intended to lyze essentially cells other than the species of interest. Such a first lysis may for example be envisioned when the biological species of interest is in a very small minority with respect to the cells of a matrix of the sample. Following the first lysis, the nucleic acids released are removed, then a second lysis is carried out, targeting the biological species of interest. In such a scenario, the control species is preferably resistant to the first lysis, and not resistant to the second lysis.

Following the lysis, DNA is extracted from the sample, for example using the extracting method described in WO2014/114896.

The DNA extracted from the sample may be essentially composed of the DNA of the matrix, i.e. of the environment from which the sample was taken. In this case, the sample may be subjected to selective capture and/or amplification, mainly targeting sequences and/or physico-chemical modifications specific to the genomes of the biological species of interest. In this case, the control species comprises the sequences and/or physico-chemical modifications targeted by the selective capture or amplification. Conversely, the sample may be subjected to a depletion essentially targeting the DNA of the matrix. In this case, the control species comprises none of the sequences or physico-chemical modifications that may be targeted by the depletion.

### Step 40: Amplification and Sequencing.

Following the extraction of DNA, the DNA fragments optionally undergo an amplification that may be of targeted type, for example via polymerase chain reaction (PCR), or of non-targeted type, for example via whole-genome amplification (WGA). The DNA extracted from the sample, where appropriate amplified, undergoes sequencing, and preferably whole-genome sequencing (WGS). Many sequencing techniques exist, for example sequencing by synthesis (SBS), or nanopore sequencing, or sequencing by hybridization. Whatever the technique employed, the aim of the sequencing is to provide digital nucleic-acid sequences, which are referred to as reads. The sequencing comprises preparing a sequencing library (library preparation), optionally followed by an amplifying step, then a step of actual sequencing. Since the technique used to sequence nucleic acid is well-known, it will not be described in detail. The amplification and sequencing may be carried out using the platform MiSeq, which is sold by the company Illumine.

During the preparation of the sequencing library, the DNA may be randomly broken up, so as to obtain nucleic-acid sequences of a targeted average length, generally an average length comprised between 50 bases and 300 bases. Reference is made to shotgun sequencing, or to whole-genome sequencing (WGS). With this type of technique, the nucleic acids, whatever their origin, are treated identically during the preparation of the sequencing library.

Following preparation of the sequencing libraries, high-throughput sequencing is carried out. The sequencer reads the bases of the sequenced DNA fragments, so as to obtain sequences that are called reads, each read corresponding to one sequence decoded by the sequencer. The sequences generated by the sequencing are then aligned with respect to genomes stored in a database, including notably the genome of the sought-after biological species of interest and the genome of the control species. Sequencing is an operation known to those skilled in the art. Details relating to sequencing operations are for example given in the documents cited with respect to the prior art, and in particular in WO2018/069430 or in the publication by Ruppé E cited above.

The sequencer transmits files, corresponding to the performed measurements and comprising the reads, to a data-processing unit. The latter comprises a memory, in which are stored instructions allowing sequencing algorithms to be implemented. The sequencing algorithms allow, for each sequence, the genome comprising the sequence to be identified among a plurality of genomes stored in a database. They also allow the position of each sequence in the genome to which it belongs to be established, and the various sequences belonging to a given genome to be assembled.

At the end of step 40, sequencing data relating to the various biological species of the sample will have been obtained. It is in particular a question of an identity of each species and of a quantity of sequences assigned to each identified species. In particular, a number R_{SOI} of sequences assigned to the biological species of interest and a number R_{SPC} of sequences assigned to the control species will have been obtained.

Step 45: Identifying the Species to which the Reads Belong.

In this step, which is implemented by the data-processing unit, the origin of each of the reads, in terms of bacterial species, is identified. This step, which is generally known as binning, or taxonomic binning, or assignment, comprises comparing each of the reads with the digital nucleic-acid sequences of a reference database. For example, Kraken, (Wood and Salzberg, “Kraken: ultrafast metagenomic sequence classification using exact alignments”, Genome Biology, 2014), or “Wowpal Wabbit” (Vervier et al., “Large-scale machine learning for metagenomics sequence classification”, Bioinformatics, 2015), or “BWA-MEM” (Li, “Aligning sequence reads, clone sequences and assembly contigs with BWA-MEM”, Genomics, 2013) are known binning software packages. Preferably, a read is assigned to a species of interest if it is entirely comprised in a genome representative of the species of interest stored in the database.

### Step 50: Normalization

The amount of sequencing data resulting from step 45 is not the same for each and every sample. Specifically, the number of sequences generated by the sequencing depends on the quality and quantity of the DNA of the various constituent biological species of the sample. It is therefore preferable, or even necessary, to normalize the quantity of sequences associated with a species with respect to a reference quantity. The normalization depends on the type of sample analyzed and on the applied metagenomic analysis. The reference quantity may for example be a total number of sequences produced for the analyzed sample. The normalized quantity of sequences associated with each species, i.e. the quantity divided by the reference quantity, is usually multiplied by 1^{E}6 so as to obtain a normalized quantity corresponding to reads per million (or RPM).

According to other variants, the reference quantity may be, non-exhaustively:
a total number of sequences associated with all the identified microorganisms;
a total number of sequences associated with an organism from which the sample was extracted: for example, when the organism is a human body, a total number of sequences associated with the human genome may be determined;
a total number of sequences associated with a reference species. By reference species, what is meant is an endogenous or exogenous species that is considered to always be present in the various samples taken. The reference species may be the control species.
a total number of sequences associated with a predetermined species in a sample not containing the biological species of interest (negative sample) or in a buffer not comprising the sample.

Step 50 is carried out for the biological species of interest (or for each biological species of interest) and for the control species (or for each control species SPC or for each calibrator). Thus, a normalized quantity RN_{SOI} is obtained for the biological species of interest SOI (or for each biological species of interest) and a normalized quantity RN_{SPC} is obtained for the control species SPC (or for each control species or for each calibrator). In the notation RN, the letter N designates the fact that the quantity is normalized.

Below, nonlimitingly, there will be considered to be only a single biological species of interest and a single control species. In the rest of the description, the term quantity may designate a normalized quantity.

### Step 60: Interpretation.

This step is an important step of the invention. It is a question of determining to what extent the results of the sequencing are interpretable.

To this end, the method comprises determining a confidence level that may be attributed to the preceding steps, and in particular to steps 30 to 50 described above. The confidence level is attributed by virtue of the control species, and in particular by virtue of the fact that the control species was introduced prior to step 30.

This step uses detection thresholds DT_{SOI} and DT_{SPC}, which are associated with the biological species of interest SOI and with the control species SPC, respectively. The detection thresholds may be established based on statistical detection thresholds determined for the biological species of interest and the control species, respectively. The statistical detection thresholds are established beforehand, in a step 100 described below. Generally, a statistical detection threshold corresponds to the lowest value, of an analyte concentration measured using a detection method, which is statistically different from the concentration measured, under the same conditions, when the analyte is absent from the sample. Each detection threshold may be equal to the statistical detection threshold, or be determined based on the statistical detection threshold, and notably be k times equal to the statistical detection threshold, k being a non-zero real number.

The interpretation aims to compare the normalized quantities RN_{SOI} and RN_{SPC} of sequences, which are assigned to the biological species of interest SOI and to the control species SPC, respectively, to their respective detection thresholds. Specifically, the biological species of interest may be considered to be detected with an acceptable confidence level when the normalized quantity of sequences assigned to the biological species of interest is higher than or equal to the detection threshold that is associated therewith. The same goes for the control species. Depending on the comparison, four situations may be distinguished between:
RN_{SOI}≥DT_{SOI} and RN_{SPC}≥DT_{SPC}: cf. step 61
RN_{SOI}≥DT_{SOI} and RN_{SPC}<DT_{SPC}: cf. step 62
RN_{SOI}<DT_{SOI} and RN_{SPC}≥DT_{SPC}: cf. step 63
RN_{SOI}<DT_{SOI} and RN_{SPC}<DT_{SPC}: cf. step 64

### Step 61 Quantification

When RN_{SOI}≥DT_{SOI} and RN_{SPC}≥DT_{SPC}, the confidence level is considered to be sufficient. Respective detections of the biological species of interest and of the control species are confirmed. The species of interest SOI is considered to be present in the sample, with a sufficient confidence level. Its concentration C_{SOI} may be estimated, on the basis of:
the concentration C_{SPC} of the control species SPC added to the sample following step 20;
the quantity R_{SPC}, optionally normalized, of sequences assigned to the control species SPC, resulting from step 45;
the number of sequences (or the normalized number of sequences) assigned to the biological species of interest, resulting from step 45;
data relating to the size of the genome of the control species and of the biological species of interest.

For example, the following expression may be used:

$${C}_{SOI} = \frac{{R}_{SOI}}{{R}_{SPC}} \times \frac{{L}_{SPC}}{{L}_{SOI}} \times {C}_{S P C} \times α \qquad ( 1 )$$

where:
L_{SPC} and L_{SOI} are the genome lengths of the control species and of the biological species of interest, respectively;
α is a correction factor determined empirically, on the basis of training samples, the concentration of biological species of interest of which is known. The correction factor a allows differences in the efficiency of the process of sequencing the biological species of interest and the control species to be taken into account. By default, α may be set equal to 1 (α=1). This unit value allows an absolute quantification to be obtained that is good enough for the positiveness or negativeness of a sample with respect to the decision threshold to be determined.

When the added concentration is expressed in GEq/mL, the concentration of the biological species of interest is also expressed in the same units.

Alternatively, the sequencing comprises assembling the sequences respectively associated with the control species and biological species of interest, and determining a coverage Coy of the assemblies for each of the species. The concentration C_{SOI} of the biological species of interest may then be computed using the following equation:

$${C}_{SOI} = \frac{C o {v}_{SOI}}{C o {v}_{SPC}} \times {C}_{S P C} \times {α}^{′} \qquad ( {1}^{′} )$$

where:
Cov_{SPC} and Cov_{SOI} are the coverages determined for the control species and the biological species of interest, respectively. Coverage expresses an average number of times a base is sequenced at a given position in the genome, as described in the publication by Lacoste C et al. “Le séquençage d'ADN à haut débit en pratique clinique” [High-throughput DNA sequencing in clinical practice], Archives de Pédiatrie 2017, 24, 373-383.
α′ is a correction factor determined empirically, on the basis of training samples, the concentration of biological species of interest of which is known. The correction factor a′ allows differences in the efficiency of the sequencing of the biological species of interest and the control species to be taken into account. By default, α′ may be set equal to 1 (α′=1). This unit value allows an absolute quantification to be obtained that is good enough for the positiveness or negativeness of a sample with respect to the decision threshold to be determined.

According to one variant described below, step 61 may be implemented with a biological species that is different from the control species and that forms a calibrator. In this case, a control species is used in step 60, to confirm the detection of the biological species of interest, while step 61, i.e. the quantification, is implemented using a calibrator, the latter being used only for the quantification. Preferably, the characteristics of the calibrator are similar to those of the control species, and correspond to the characteristics described with reference to step 20. The quantification, using the calibrator, may be carried out using expression (1) or expression (1′). Expression (1) becomes:

$${C}_{SOI} = \frac{{R}_{SOI}}{{R}_{CAL}} \times \frac{{L}_{CAL}}{{L}_{SOI}} \times {C}_{C A L} \times α \qquad ( {1}^{″} )$$

where:
R_{CAL} is the preferably normalized number of sequences assigned to the calibrator;
L_{CAL} is the length of the genome of the calibrator;
C_{CAL} is the calibrator concentration added to the sample;
α is a correction factor such as described with reference to (1).

Expression (1′) becomes:

$${C}_{SOI} = \frac{C o {v}_{SOI}}{C o {v}_{CAL}} \times {C}_{C A L} \times {α}^{′} \qquad ( {1}^{′′′} )$$

Cov_{CAL} is a coverage determined for the calibrator;
α′ is a correction factor such as described with reference to (1′).

According to one embodiment, no control species is used. According to this embodiment, a calibrator is used, and the concentration of the biological species of interest is employed based on the, preferably normalized, number of sequences.

### Step 62:

When RN_{SOI}≥DT_{SOI} and RN_{SPC}<DT_{SPC}, this means that the control species is considered not detected whereas the biological species of interest is considered detected. However, the biological species of interest cannot be quantified with sufficient confidence. The confidence level is considered to be insufficient. This step comprises comparing the added concentration C_{SPC} of the control species and the decision threshold SD, such that:
if C_{SPC}<SD, no information can be obtained on the concentration of biological species of interest relative to the decision threshold;
if C_{SPC}≥SD, the concentration of biological species of interest cannot be estimated, but it may be considered to be higher than the decision threshold. Although it is not possible to quantify the concentration of the biological species of interest, it is possible to conclude that the decision threshold has been crossed.

### Step 63:

When RN_{SOI}<DT_{SOI} and RN_{SPC}≥DT_{SPC}, the sequencing may be considered to have worked correctly. The confidence level is considered to be sufficient. This step comprises estimating a minimum detectable concentration of the biological species of interest. The minimum detectable concentration Cmin_{SOI} of the biological species of interest corresponds to the lowest concentration able to be distinguished from background noise. It is comparable to the concentration, in genome equivalent, corresponding to the detection threshold DT_{SOI} of the biological species of interest. The minimum detectable concentration may be determined on the basis:
of the concentration C_{SPC} of the control species SPC added to the sample following step 20;
of the number R_{SPC} of sequences assigned to the control species SPC, resulting from step 45;
of the detection threshold DT_{SOI} associated with the biological species of interest;
of data relating to the size of the genome of the control species and of the biological species of interest.

$$C {min}_{SOI} = \frac{D {T}_{SOI}}{{R}_{SPC}} \times \frac{{L}_{SPC}}{{L}_{SOI}} \times {C}_{S P C} \times α \qquad ( 2 )$$

where:
L_{SPC} and L_{SOI} are the genome lengths of the control species SPC and of the biological species of interest SOI, respectively;
α is the correction factor described with reference to equation (1).

Step 63 comprises comparing the decision threshold SD to the minimum detectable concentration Cmin_{SOI}, such that:
if Cmin_{SOI}≤SD, detection of the biological species of interest may be considered to be negative: the concentration of biological species of interest in the sample is lower than or equal to the decision threshold;
if Cmin_{SOI}>SD, no information can be provided on the presence of the biological species of interest in the sample and on its concentration with respect to the decision threshold.

### Step 64:

When RN_{SOI}<DT_{SOI} and RN_{SPC}<DT_{SPC}, the absence of detection of the control species SPC suggests that the analysis has not achieved the performance required for detection of the biological species of interest. The confidence level is considered to be insufficient. The analysis cannot be interpreted. The analysis may be considered to be invalid. Such a situation may arise:
when one of the steps of the sequencing does not achieve the performance required for detection of the biological species of interest;
and/or when the sample comprises a high quantity of DNA of the patient or of the matrix or of microbiological flora;
and/or when the sample comprises at least one species with a high concentration, and that generates a high number of sequences, this having the effect of masking other sequences of interest.

At the end of one of steps 61 to 64, the confirmation of the presence of the biological species of interest, in a concentration higher than the decision threshold, and its quantification if any, are used to assist with diagnosis.

### Variant

In the embodiment described above, the control species SPC performs both a function regarding control of the quality of the metagenomic analysis and a calibrator function, allowing the biological species of interest in the sample to be quantified.

According to one variant, a control species SPC and a calibrator that is different from the control species, are added to the sample. It is for example a question of two different bacterial species. The control species SPC performs a function regarding control of the quality of the metagenomic analysis. The calibrator allows the biological species of interest in the sample to be quantified, according to equation (1) or (1′) or (2). When it is different from the control species, the calibrator preferably has the same characteristics as the control species, these characteristics being described with reference to step 20. The control species SPC is added in a first concentration. A detection threshold is allocated thereto and step 60 is implemented by comparing a normalized quantity of sequences assigned to the control species, which results from step 50, to the detection threshold associated with the control species. The calibrator is also added to the sample, in a second concentration. A detection threshold is allocated thereto. In step 61, the quantification may be carried out taking into account a normalized quantity of sequences associated with the calibrator, and the detection threshold that is associated therewith.

The calibrator may be added prior to the lysis or following the lysis and prior to the sequencing.

In another variant, a plurality of calibrators are added to the sample, each calibrator being chosen for one or more species of interest. In particular, groups of bacterial species may react substantially differently to the processes of extracting nucleic acids (for example Gram+ bacteria and Gram− bacteria). Advantageously, a calibrator consisting of a Gram+ bacterium is added when one or more species of interest are Gram+ and a calibrator consisting of a Gram− bacterium is added when one or more species of interest are Gram−. Similarly, the species of interest may consist of bacteria and viruses. In this case, a first calibrator is bacterial and a second calibrator is viral. auxiliary is viral. Generally, it is a question of choosing a calibrator that behaves, in the steps of sample preparation (extraction, optionally sequence library preparation or amplification and sequencing), as identically as possible to the species of interest that it calibrates.

### Step 100: Establishing the Detection Thresholds.

As mentioned above, it is necessary for the control species and the biological species of interest to respectively be associated with detection thresholds. For a given biological species (control biological species or biological species of interest), the detection threshold is established prior to the interpretation of the results, using training samples not comprising said species. It is a question of samples that are negative relative to the species in question. These samples are representative of the analyzed sample. By representative, what is meant is that these training samples comprise a population of biological species that is comparable to that of the analyzed sample, both from a qualitative and from a quantitative point of view. The absence of the biological species of interest and/or of the control species from each test sample may be verified using a standard culture- and/or PCR-based method.

On each training sample, sequencing is carried out, preferably under the same conditions as described with reference to steps 30 to 45. Following the sequencing, a quantity of sequences assigned to the species in question is determined. This quantity is preferably normalized, as described with reference to step 50.

Thus, the detection thresholds respectively associated with the biological species of interest and with the control species may be established using first training samples, not comprising the biological species of interest, and second training samples, not comprising the control species, respectively. The first training samples may be none other than the second training samples, and vice versa, in which case the detection thresholds associated with the biological species of interest and with the control species are determined with the same training samples.

The sequencing is preferably carried out on a statistically representative number of training samples. Thus, a statistical distribution of the normalized quantity of sequences is obtained. Next, a mean μ of the distribution, and a dispersion indicator, for example the standard deviation σ or variance σ², are estimated. The detection threshold is estimated by adding, to the mean μ, n times the dispersion indicator, n being a real number. n is typically comprised between 2 and 4.

Since the detection thresholds respectively associated with the biological species of interest and with the control species are intended to be compared to normalized quantities of sequences of the biological species of interest and of the control species, it is important for the normalization carried out in step 100 to be similar to the normalization carried out in step 50.

The steps described above may simultaneously target a plurality of biological species of interest. This is moreover a notable advantage of metagenomic analysis, which allows various biological species to be addressed simultaneously. Another advantage of metagenomic analysis is the ability to use a plurality of control species simultaneously. Thus, one control species may be used to target one or more biological species, whereas another control species may be used to target other biological species of interest. This is another advantage of metagenomic analysis.

It is even envisionable to use a plurality of control species for a given biological species of interest. For example, steps 61 to 64 may be implemented using, for a given biological species of interest, various control species. This makes it possible to limit the risk of the method failing due to defective sequencing of a control species. An estimate as to the presence of the biological species of interest with respect to the decision threshold is obtained for various (biological species, control species) pairs. When a plurality of control species are used for a given biological species of interest, it is possible to obtain a plurality of quantifications, according to equations (1), (1′), in which case the mean or median of the obtained quantifications, or the quantification considered to be the most penalizing, i.e. the quantification leading to the highest concentration of biological species of interest, or, more generally, the concentration closest to the decision threshold, may be considered.

More generally, metagenomic analysis still requires powerful computing means. In contrast, it permits a certain degree of operating flexibility, in that it allows a plurality of biological species (and/or a plurality of control species) to be addressed simultaneously, the only condition being that the genome of the sought-after biological species and the genome of their respective control species must be known.

Steps 61 to 64 are implemented by a computing unit, a microprocessor for example, on the basis of sequencing data generated in steps 40, 45 and 50 and delivered by the processing unit. The sequencing data, which correspond to measured data obtained from the analysis sample, are thus transmitted, via a wired or wireless link, to the computing unit, so that one of steps 61 to 64 may be executed. The microprocessor is connected to a memory containing instructions allowing steps 61 to 64 to be implemented.

### Example 1

In a first example, it was verified that Bacillus subtilis is a good candidate for use as control species in metagenomic sequencing of samples resulting from bronchoalveolar lavages (BALs) carried out on human patients. As the patient is human, this type of sample is expected to comprise a high quantity of human DNA.

Metagenomic sequencing of such samples may make it possible to assist with diagnosis of hospital-acquired pneumonias, for diagnostic purposes. The clinical decision threshold was set to 1.0 E4 CFU/mL, CFU being the acronym of colony forming unit.

In order to remove the DNA of the patient, the analysis protocol comprised a preliminary lysis in which the DNA of the patient was removed. In a first lysis, the sample was treated with a lysing agent that specifically targeted the cells of the patient. Such a lysing agent is for example described in WO2014/114896. The DNA released was then removed via enzymatic action and washing. The sample then underwent a second mechanical and chemical lysis to extract bacterial DNA.

Prior to the lysing steps, provision was made in the protocol to add a control species to the sample. The biological species forming the control species had to be resistant to the lysis of the human cells, while being sensitive to the lysis of the bacterial cells. Now, it is known that certain bacteria, in particular Gram-positive bacteria, are difficult to lyze. Therefore, a biological species having a lysis resistance equivalent to that of a Gram-positive bacteria was chosen by way of control species.

Moreover, the metagenomic sequencing carried out aimed to detect and potentially quantify about 20 biological species of interest, each species of interest being a bacterium contained in the following list: Acinetobacter baumannii, Citrobacter freundii, Citrobacter koseri, Enterobacter aerogenes, Enterobacter cloacae, Escherichia coli, Haemophilus influenzae, Hafnia alvei, Klebsiella oxytoca, Klebsiella pneumoniae, Legionella pneumophila, Morganella morganii, Proteus mirabilis, Proteus vulgaris, Providencia stuartii, Pseudomonas aeruginosa, Serratia marcescens, Staphylococcus aureus, Stenotrophomonas maltophilia, Streptococcus pneumoniae.

The control species SPC also had to be able to be sequenced with an efficiency comparable to the species of interest listed above. It is known that sequencing efficiency essentially depends on the size of the genome and on GC (Guanine—Cytosine) content. Thus, in this example, the control species had to have a genome size comprised between 1.9 and 6.6 megabases, and a GC content comprised between 33% and 66%. Moreover, the concentration of the control species, added to the sample, was set to 1.0 E4 CFU/mL, i.e. to a concentration comparable to the aforementioned decision threshold.

The inventor evaluated the desirability of using the following biological species to form the control species: Bacillus stearothermophilus, Synechocystis sp. PCC6803, Pelagibacter ubique, Methanocaldococcus jannaschii, Aeropyrum pernix, Kocuria rhizophila, Azospirillum lipoferum, Lactococcus lactis, Synechococcus sp. WH 7805, Schizosaccharomyces pombe, Pantoea stewartii, Phage T4, Pichia pastoris, Armored DNA Quant™ and Bacillus subtilis.

Among these various species, it turned out that Bacillus subtilis had the characteristics required to be used as control species. The size of the genome of Bacillus subtilis is 4.12 Mb (megabases) and it has a GC content of 43.6%. In addition, Bacillus subtilis is commercially available in the form of “BioBalls” (registered trademark)—manufacturer Biornerieux. These BioBalls are water-soluble balls containing a calibrated concentration of Bacillus subtilis, this allowing the concentration of the control species added to be adjusted. Rehydration of a BioBall MultiShot 550 in a bronchoalveolar-lavage sample of 600 μL corresponded to an added concentration of Bacillus subtilis equal to 9.2 E3 CFU/mL, this being close to the decision threshold of 1.0 E4 CFU/mL.

DNA extracts from samples comprising fresh cultures of Bacillus subtilis and from samples comprising Bacillus subtilis added in the form of BioBalls were also compared by real-time PCR. The results of the PCRs were comparable.

7 samples obtained by bronchoalveolar lavage (BAL) were sequenced, without prior addition of Bacillus subtilis. In 4 of the 7 samples, the number of sequences assigned to Bacillus subtilis was observed to be negligible: lower than 5 reads per million. Thus, the number of false positives was negligible. In the other samples, sequences were assigned to Bacillus subtilis either as a result of a sequence-assigning software error, or as a result of the presence of sequences very similar to those of Bacillus subtilis in the sample. However, the number of sequences assigned to Bacillus subtilis was never more than 200 reads per million: it was thus relatively low.

46 samples obtained by BAL had Bacillus subtilis added in a concentration of 1.7 E4 CFU/mL, to within an uncertainty. After sequencing, the number of sequences assigned to Bacillus subtilis exceeded 1000 reads per million for 36 of the 46 samples.

This example shows that Bacillus subtilis is a biological species apt to form a control species, in a sample obtained by BAL, and with the analysis protocol described at the start of the example.

### Example 2

This example describes detection and quantification of Staphylococcus aureus in a sample obtained by bronchoalveolar lavage (BAL) with application of the double-lysis protocol described in example 1 and steps 10 to 50 described above.

A cohort of 13 samples obtained by BAL was used. Based on the conclusions of example 1, the control species used was Bacillus subtilis, which was added to each sample in a concentration close to the decision threshold (1.0 E4 CFU/mL). In this example, the control species was obtained by rehydration of a BioBall MultiShot 10^{E}8-Bacillus subtilis ATCC 19659 (Biornerieux), in 1.1 mL of PBS buffer (PBS standing for phosphate-buffered saline). The control species was diluted to 1.0 E6 CFU/mL in PBS and 10 μL added to 600 μL of sample. Thus, an added concentration of the control species of 1.7 E4 CFU/mL was obtained.

Each sample was treated at most 48 hours after the sample was taken. As indicated above, each sample underwent a first lysis specific to the human cells. Unlyzed cells were pelleted and treated in DNase I. Before extraction of the human DNA, the DNase was deactivated by heating and adding EDTA (ethylenediaminetetraacetic acid). Each sample was then subjected to a second lysis, which was performed by adding the sample to a bead-beating tube containing a mixture of glass beads of 1 mm diameter and of Zr/Si beads of 0.1 mm diameter. The lysis was obtained by shaking the tube for 20 minutes. The DNA was extracted from the lysate using the Biornerieux platform easyMAG (registered trademark). Elution was carried out in a volume of 25 μL. The extracts were stored at −20° C.

A sequencing library for 2×250 paired-end reads was prepared with the Nextera (registered trademark) XT DNA Library Preparation Kit (manufacturer Illumine). The samples were sequenced using the MiSeq (registered trademark) platform with the “MiSeq reagent kit V3” (Illumine).

The sequences were processed with a processing unit using the software package KRAKEN VO 10.5b and an internal sequence database. This database contained, notably, the sequences of the human genome and the sequences of 20 biological species of interest, which were listed in example 1. The number of sequences produced in each sample varied between 331 000 and 17 000 000. The numbers of sequences associated with the control biological species (Bacillus subtilis) and the biological species of interest (S. Aureus) were normalized to reads per million (RPM).

Moreover, quantitative reference measurements were carried out, on each sample, by quantitative PCR (qPCR), targeting the SpA gene. Amplification and real-time read-out of the fluorescent signal were carried out on the platform CFX96 Touch Real-Time PCR Detection System (Biorad).

Table 1 collates the results of the sequencing for 13 culture-positive samples. Columns 1 to 7 respectively correspond:
to the reference of the sample;
to a quantification of S. aureus by culture;
to a quantification of S. aureus by qPCR,
to the normalized quantity RN_{SPC} of sequences assigned to the control species (B. subtilis);
to the normalized quantity RN_{SOI} of sequences assigned to the biological species of interest (S. aureus);
to a quantification, when one was possible, of the concentration C_{SOI} of the biological species of interest determined using equation (1), which was described in step 61;
to a quantification, when one was possible, of the concentration C_{SOI} of the biological species of interest determined using equation (1′), which was described in step 61.

In this example, the control species SPC played the role of calibrator, in the sense that it was used in the quantifying step.

SOI NA and SPC NA correspond to the fact that the number of sequences associated with the biological species of interest SOI and with the control species SPC, respectively, was insufficient to allow assembly. NA is the acronym of Not Assembled.

TABLE 1

| Sam- | Culture | qPCR | RN_{SPC} | RN_{SOI} | C_{SOI} (1) | C_{SOI} (1)′ |
|---|---|---|---|---|---|---|
| ple | CFU/mL | GEq/mL | (RPM) | (RPM) | GEq/mL | GEq/mL |
| 1 | 1^{E}6 | 1.6^{E}7 | 737 | 824740 | 2.7^{E}7 | 2.0^{E}6 |
| 2 | 1^{E}3 | 1.9^{E}6 | 187 | 11080 | 1.4^{E}6 | SPC NA |
| | | | | | | SOI NA |
| 3 | >1^{E}5 | 1.8^{E}6 | 48 | 4418 | 2.2^{E}6 | SPC NA |
| 4 | 1^{E}5 | 3.1^{E}5 | 1255 | 98109 | 1.9^{E}6 | 3.0^{E}5 |
| 5 | 1^{E}2 | 2.0^{E}4 | 398 | 2256 | 1.4^{E}5 | SPC NA |
| 6 | 1^{E}5 | 4.2^{E}5 | 3605 | 129716 | 8.7^{E}5 | 2.3^{E}5 |
| 7 | >1^{E}5 | 9.6^{E}4 | 116 | 1793 | 3.8^{E}5 | SPC NA |
| 8 | 1^{E}5 | 3.3^{E}4 | 0 | 74 | Invalid | Invalid |
| 9 | 1^{E}5 | 2.9^{E}4 | 1225 | 4956 | 9.8^{E}4 | 1.6^{E}4 |
| 10 | 1^{E}5 | 1.5^{E}5 | 1681 | 64201 | 9.3^{E}5 | 5.6^{E}4 |
| 11 | 1^{E}4 | 8.8^{E}5 | 706 | 40714 | 1.4^{E}6 | 9.7^{E}4 |
| 12 | 1^{E}4 | 4.4^{E}3 | 9302 | 2054 | 5.3^{E}3 | 1.0^{E}4 |
| 13 | 1^{E}2 | 9.5^{E}2 | 272 | 3 | 2.7^{E}2 | SOI NA |

Samples 1, 2, 3, 4, 5, 6, 7, 9, 10, 11, 12 and 13 (i.e. 12 samples out of 13) correspond to the configuration described with reference to step 61, in which a quantification of the species of interest is possible, for example according to expression (1) and expression (1′).

Sample 8 corresponds to the configuration described with reference to step 64: the results are not interpretable. Additional investigations revealed, for this sample, that the sequence-demultiplexing step failed. This particular case is interesting, because it shows that taking into account the control species allowed generation of a “false negative” to be avoided.

For the samples that were “quantifiable” (1, 2, 3, 4, 5, 6, 7, 9, 10, 11, 12 and 13), the concentration C_{SOI} was estimated using equation (1′). However, the sequences associated with the control species SPC or with the biological species of interest SOI were sometimes not assemblable; in this case, the biological species of interest was not quantifiable using this protocol, whereas it was using equation (1). This was notably the case for samples 2 and 13, in which the quantities of sequences associated with the biological species of interest were insufficient to obtain assembly and to measure a sequencing depth. Thus, quantification based on equation (1′) is envisionable only when the quantity of sequences is sufficient. A quantification based on equation (1) seems preferable.

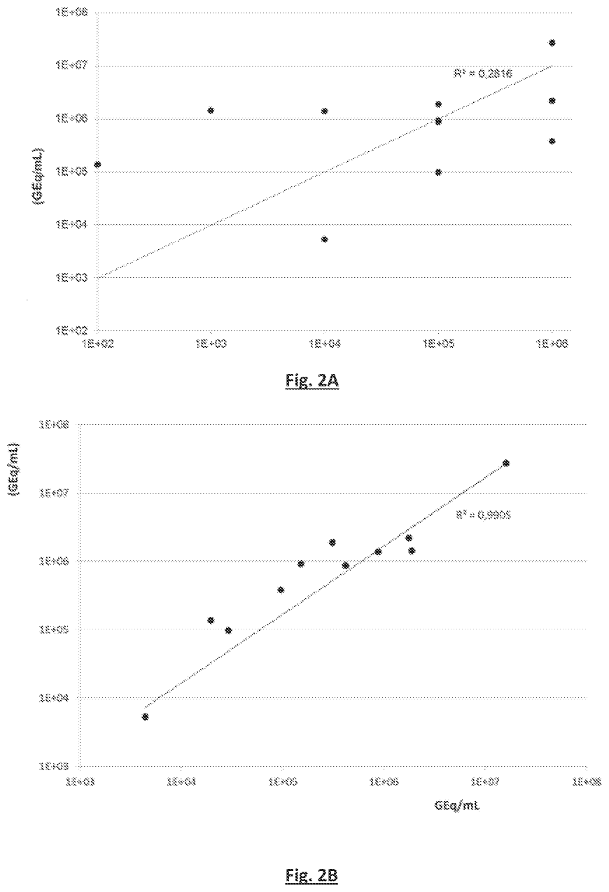

FIG. 2A shows a comparison of the quantification of S.aureus by culture (x-axis) and by sequencing (y-axis). The correlation coefficient is low (r²=0.2929). This low value is explicable by the imprecision of the culturing method, and by the difference between the quantity of viable and cultivatable cells, which are detected by culture, and the total quantity of genomes, which is detected by sequencing. Certain patients from whom samples were taken were being treated with antibiotics, this tending to decrease the proportion of viable and cultivatable bacteria with respect to the total number of bacteria. Thus, culture allows only partial quantitative information to be obtained.

FIG. 2B shows a correlation between the results of quantification by meta-sequencing (equation (1)—y-axis) and by quantitative PCR (x-axis). The correlation coefficient is higher: r²=0.9906, this demonstrating the reliability of the quantification by meta-sequencing.

### Example 3

In this example, detection of 20 pathogenic bacterial species of interest, which species were listed in example 1, in samples obtained by bronchoalveolar lavage (BAL) or mini-bronchoalveolar lavage (mini-BAL), was tested. The control species SPC (B. subtilis) was obtained in the same way as in example 2, the concentration added to each sample being 1.7 E4 CFU/mL. The decision threshold was 1.0 E4 CFU/mL for BAL samples, and 1.0 E3 CFU/mL for mini-BAL samples.

Two cohorts of samples were collected: one training cohort, comprising 46 samples (23 BAL and 23 mini-BAL samples), and one analysis cohort, comprising 40 samples (33 BAL and 7 mini-BAL samples).

For all of the samples of the training and analysis cohorts, culture reference measurements were taken for each species of interest.

The sample underwent a double lysis, as described in example 2. The sequencing was carried out as described in example 2.

For each species of interest, and for the control species, the quantity of sequences was normalized to reads per million of reads associated with the bacterial species (RPMb), cf. step 50.

For each of the biological species of interest, the detection threshold DT_{SOI} was determined considering only training samples for which the biological species of interest was considered not detected. The species of interest was considered not detected in a sample when the result of microbiological culture of the sample was negative in respect of detection of the SOI in question and negative in respect of detection of MetaPhlAn marker sequences specific to the SOI in question. FIG. 3 shows the statistical distributions of normalized sequence quantities in training samples that were negative in respect of the species of interest. The x-axis corresponds to each species of interest, whereas the y-axis corresponds to the normalized quantity of sequences associated with the species of interest. For each species, the median value (line contained in the box), and the 25th and 75th percentiles (limits of the box) were determined, this allowing a representation in the form of a box-and-whisker plot (or box plot) to be obtained. The ends of each vertical line correspond to the 1st and 99th percentiles. It may be seen that the distributions vary greatly with respect to one another, this justifying the use of one detection threshold DT_{SOI} per biological species of interest. For each of the species of interest, a detection threshold DT_{SOI} was determined, according to step 100 described above. If μ_{SOI} designates the mean of the normalized number of sequences assigned to the species of interest, and σ_{SOI} is their standard deviation, the detection threshold DT_{SOI} is placed “3-sigma” above the mean, according to the expression:

DT_{SOI}=μ_{SOI}+3σ_{SOI} (3)

The detection threshold DT_{SPC}=DT_{B. subtilis} associated with B. subtilis was defined. 7 training samples to which no B. subtilis was added were taken into account. The mean μ_{B. subtilis} of the normalized number of sequences assigned to B. subtilis, and their standard deviation σ_{B. subtilis}, were determined. The detection threshold DT_{B. subtilis} is such that:

DT_{B.subtilis}=μ_{B.subtilis}+3σ_{B.subtilis} (3)

A decision threshold (SD), referred to as the metagenomic threshold, was defined in order to distinguish between a normal presence of bacteria of interest and infections of patients by these bacteria of interest. To this end, the results of microbiological cultures of the samples of the training cohort were divided into 2 separate populations:
the “infection” population corresponded to 20 occurrences of detection by culture in concentrations equal to or higher than clinical thresholds, namely 1.0 E3 CFU/mL for the mini-BAL samples and 1.0 E4 CFU/mL for the BAL samples.
the “colonization” population corresponded to 900 occurrences of non-detection by culture or of detection by culture in concentrations lower than clinical thresholds, namely 1.0 E3 CFU/mL for the mini-BAL samples and 1.0 E4 CFU/mL for the BAL samples.

In the two preceding paragraphs, the 920 occurrences corresponded to analyses, by micro-culture, of the 46 training samples, carried out with respect to each of the 20 biological species of interest.

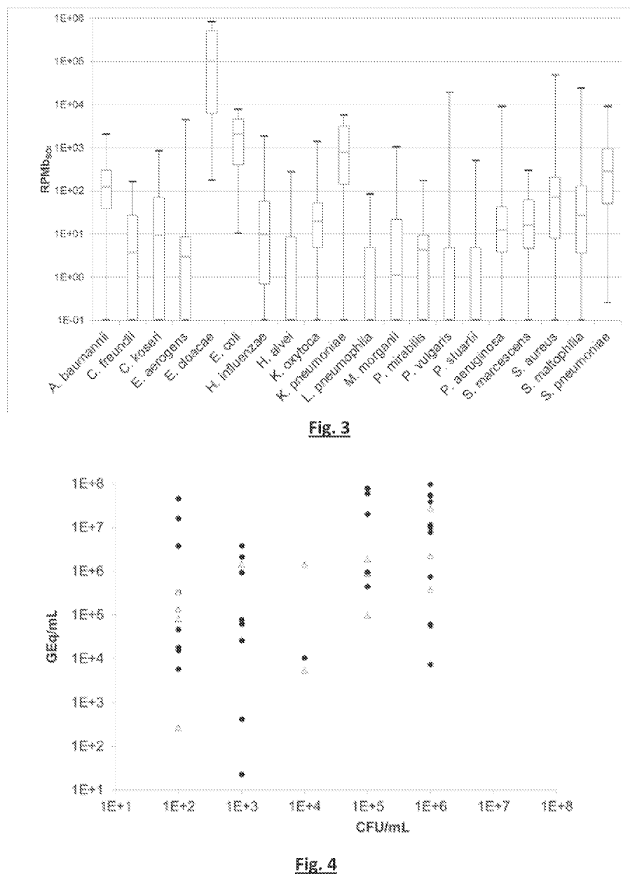

FIG. 4 shows, for various samples, quantifications of biological species carried out by culture (x-axis) and by metagenomic analysis (y-axis). In FIG. 4, the black circles correspond to a species chosen from Acinetobacter baumannii, Citrobacter freundii, Citrobacter koseri, Enterobacter aerogenes, Escherichia coli, Haemophilus influenzae, Hafnia alvei, Klebsiella oxytoca, Klebsiella pneumoniae, Legionella pneumophila, Morganella morganii, Proteus mirabilis, Proteus vulgaris, Providencia stuartii, Pseudomonas aeruginosa, Serratia marcescens, Stenotrophomonas maltophilia and Streptococcus pneumoniae. The white triangles correspond to Staphylococcus aureus.

Although, as shown in example 2 (FIG. 2A), it is sometimes not possible to precisely correlate the concentration in CFU/mL obtained by culture and the concentration in GEq/mL obtained by meta-sequencing, FIG. 4 shows that, for a species of interest, or fora group of species of interest, the “colonization” and “infection” populations may nonetheless be differentiated between on the basis of the results (in genome equivalent (GEq)) of quantification by sequencing. The metagenomic threshold (SD) was defined taking into account the first half centile of the concentrations measured in the “infection” population; the value thus obtained was 5.5 ^{E}3 GEq/mL.

Thus, on the basis of training samples, it is possible to define a metagenomic threshold that forms a decision threshold SD allowing samples having a concentration of biological species of interest that is located above or below a critical value to be separated. The critical value may notably correspond to the decision threshold SD described above. The concentration of a species of interest, determined by sequencing, was then compared to the decision threshold associated therewith. It will be noted that the decision threshold generally depends on the biological species in question. It is thus possible to establish one decision threshold for one biological species in question or for one group of biological species. Two different biological species may be associated with two different decision thresholds.

The 40 samples of the analysis set were sequenced. Tables 2A to 2C collate the obtained results, each table collating the results of samples 1 to 13, 14 to 27 and 28 to 40, respectively. The first row of each table contains the reference of each sample. The second row represents detection (+) or non-detection (−) of the control species SPC with respect to the detection threshold DT_{SPC} that is associated therewith: cf. step 60.

In samples 3, 7, 23 and 35, the control species SPC was not detected (RN_{SPC}<DT_{SPC}). When the species of interest was not detected (RN_{SOI}<DT_{SOI}), cf. step 64, the results were not interpretable, this corresponding to the code INV. It was not possible to determine the concentration of the species of interest with respect to the decision threshold, in the present case the clinical threshold, due to the minimum detectable concentration being too high. When the species of interest was detected (RN_{SOI}≥DT_{SOI}), cf. step 62, because the control biological species was added in a concentration higher than the metagenomic threshold (SM), which was equal to 5.5 ^{E}3 GEq/mL, detection of the species of interest SOI was considered to be positive above the decision threshold, which in this example is a clinical decision threshold. This result corresponds, in tables 2A, 2B and 2C:
either to a true positive (TP) when the biological species of interest is also detected to be above the clinical threshold by microbiological culture;
or to a false positive (FP or FP+) when the biological species of interest is not detected to be above the clinical threshold by microbiological culture.

In samples 1, 2, 4-7, 8-22, 24-34 and 36-40, the biological control species was detected (RN_{SPC}≥DT_{SPC}). When the species of interest was not detected (RN_{SOI}<DT_{SOI}), cf. step 63, the minimum detectable concentration Cmin_{SOI} was established using equation (2). When the minimum detectable concentration Cmin_{SOI} was higher than the decision threshold SD, these results were not interpretable, this corresponding to the code INV in tables 2A, 2B and 2C. When the minimum detectable concentration Cmin_{SOI} was lower than or equal to the decision threshold (metagenomic threshold) SD, the detection of the biological species of interest was considered to be lower than the clinical threshold. This result corresponds, in tables 2A, 2B and 2C:
to a false negative (FN) when the biological species of interest is detected to be above the clinical threshold by microbiological culture, but quantified to be below the decision threshold by the metagenomic analysis.
to true negatives (empty boxes) when the biological species of interest is not detected to be above the clinical threshold by microbiological culture and by the metagenomic analysis.

When the biological control species was detected (RN_{SPC}≥DT_{SPC}), and the biological species of interest was detected (RN_{SOI}≥DT_{SOI}), the number of sequences associated with the biological species of interest was used as calibrator to establish the concentration C_{SOI} of the biological species of interest, using expression (1) described in step 61. These results correspond, in tables 2A, 2B and 2C:
to a true positive (TP) when the biological species of interest is detected to be above the clinical threshold by microbiological culture;
or to a false positive (FP or FP+) when the biological species of interest is not detected to be above the clinical threshold by microbiological culture.

TABLE 2A

| Sample | 1 | 2 | 3 | 4 | 5 | 6 | 7 | 8 | 9 | 10 | 11 | 12 | 13 |
|---|---|---|---|---|---|---|---|---|---|---|---|---|---|
| SPC | + | + | − | + | + | + | − | + | + | + | + | + | + |
| A. baumannii | | | INV | | | | INV | INV | | | | | |
| C. freundii | | | INV | | | | INV | | | | | | |
| C. koseri | | | INV | | | | INV | INV | | | | | |
| E. aerogens | | | INV | INV | | INV | INV | INV | | | | | |
| E. cloacae | INV | INV | INV | INV | INV | INV | INV | INV | INV | INV | INV | INV | INV |
| E. coli | | | INV | INV | | INV | INV | INV | | | | | |
| H. influenzae | | | INV | | | INV | INV | INV | | | | | |
| H. alvei | | | INV | | | | INV | | | | | | |
| K. oxytoca | | | INV | | | | INV | INV | | | | | |
| K. pneumoniae | | | INV | INV | | INV | INV | INV | | | | | |
| L. pneumophila | | | INV | | | | INV | | | | | | |
| M. morganii | | | INV | | | | INV | | | | | | |
| P. mirabilis | | | INV | | | | INV | | | | | | |
| P. vulgaris | | | INV | INV | | INV | INV | INV | INV | | | | INV |
| P. stuartii | | | INV | | | | INV | | | | | | |
| P. aeruginosa | | | TP | | | FP | INV | | | | | | |
| S. marcescens | | | INV | | | FP+ | FP+ | | | | | | |
| S. aureus | | | INV | INV | | INV | INV | INV | | | | | TP |
| S. maltophilia | | | INV | | | | INV | INV | | | | | |
| S. pneumoniae | TP | | INV | INV | | INV | INV | INV | TP | | | | |

TABLE 2B

| Sample | 14 | 15 | 16 | 17 | 18 | 19 | 20 | 21 | 22 | 23 | 24 | 25 | 26 |
|---|---|---|---|---|---|---|---|---|---|---|---|---|---|
| SPC | + | + | + | + | + | + | + | + | + | − | + | + | + |
| A. baumannii | | | | | | | | | INV | INV | | | |
| C. freundii | | | | | | | | | | INV | | | |
| C. koseri | | | | | | | | | INV | INV | | | |
| E. aerogens | | | | | | | | | INV | INV | | | |
| E. cloacae | INV | INV | INV | INV | INV | INV | INV | INV | INV | INV | INV | INV | INV |
| E. coli | | | | | | INV | | | INV | INV | | | |
| H. influenzae | | | | | | | | | INV | INV | | | |
| H. alvei | | | | | | | | | | INV | | | |
| K. oxytoca | | | | | | | | | INV | INV | | | |
| K. pneumoniae | | | | | | | | | INV | INV | | | |
| L. pneumophila | | | | | | | | | | INV | | | |
| M. morganii | | | | | | | | | INV | INV | | | |
| P. mirabilis | | | | | | | | | | INV | | | |
| P. vulgaris | | | | | | INV | INV | | INV | INV | | | |
| P. stuartii | | | | | | | | | INV | INV | | | |
| P. aeruginosa | | | | | | | TP | | INV | TP | | | |
| S. marcescens | | | | | | | | | | INV | | | |
| S. aureus | | | | | | | | | INV | INV | | | |
| S. maltophilia | | | | | | | | | TP | INV | | | |
| S. pneumoniae | | | | | | FP | | | INV | INV | | | |

TABLE 2C

| | 27 | 28 | 29 | 30 | 31 | 32 | 33 | 34 | 35 | 36 | 37 | 38 | 39 | 40 |
|---|---|---|---|---|---|---|---|---|---|---|---|---|---|---|
| SPC | + | + | + | + | + | + | + | + | − | + | + | + | + | + |
| A. baumanii | INV | | INV | | | | | | INV | | | | INV | |
| C. freundii | FP | | | | | | | | INV | | | | | |
| C koseri | INV | | | | | | | | INV | | | | INV | |
| E. aerogens | INV | | FP+ | | | | | | INV | | | | INV | |
| E. cloacae | INV | INV | INV | INV | INV | INV | | INV | INV | INV | INV | INV | INV | INV |
| E. coli | INV | | INV | | | | | INV | INV | | INV | | INV | |
| H. influenzae | INV | | INV | | TP | | | | INV | | | | INV | |
| H. alvei | FP | | | | | | | | INV | | | | | |
| K. oxytoca | FP | | FP | | | | | | INV | | | | INV | |
| K. pneumoniae | FP+ | | INV | | | | | INV | INV | | | | INV | |
| L. pneumophila | | | | | | | | | INV | | | | | |
| M. morganii | INV | | | | | | | | INV | | | | INV | |
| P. mirabilis | | | | | | | | | INV | | | | | |
| P. vulgaris | INV | | INV | | | | | INV | INV | | INV | | INV | |
| P. suartii | INV | | | | | | | | INV | | | | | |
| P. aeruginosa | INV | | INV | | | | | | TP | | | | TP | FP |
| S. marcescens | FP | FP | | | | | | FP | INV | | | | | |
| S. aureus | INV | INV | FP+ | | INV | | | INV | INV | | INV | INV | INV | |
| S. maltophilia | INV | | INV | | | | | INV | INV | | INV | FP | INV | |
| S. pneumoniae | INV | | INV | | | | | INV | INV | | INV | | INV | |

Analysis by microbiological culture allowed 11 occurrences above the decision threshold (1^{E}4 CFU/mL for the BAL samples and 1^{E}3 CFU/mL for the mini-BAL samples) to be detected. The metagenomic analysis allowed 10 of these occurrences to be detected, this corresponding to the notation TP (true positive) in tables 2A to 2C. The occurrence not detected by metagenomics corresponded to E. cloacae in sample 27 and was explicable by the high quantity of sequences that was associated with E. cloacae in samples from which this bacterium was absent (see FIG. 3), this leading to a very high detection threshold, which resulted in the minimum detectable concentration Cmin_{SOI} frequently being higher than the metagenomic threshold (SM). This result was considered by the metagenomic test to be invalid, cf. INV in table 2C.

The metagenomic analysis allowed 19 additional occurrences to be detected, with respect to microbiological culture. These occurrences are designated FP (false positive) or FP+ in tables 2A to 2C. The 5 FP+ occurrences corresponded to detections for which MetaPhlAn markers and BLAST alignments (BLAST being the acronym of Basic Local Alignment Search Tool) allowed the presence of the species of interest in the sample to be confirmed, despite its non-detection by culture. These complementary occurrences were probably due to a better sensitivity of the metagenomic test with respect to the detection by microbiological culture, which allowed only detection of the viable and cultivatable part of the microbiota. The FP occurrences corresponded to false positives for which the number of reads associated with the species of interest was too low for a confirmation to be possible via a search for MetaPhlAn markers and BLAST alignments. These complementary occurrences were also probably due to a better sensitivity of the metagenomic test with respect to the detection by microbiological culture; however, the absence of confirmation prevents a lack of specificity of the metagenomic test from being ruled out.

The metagenomic test generated 185 invalid results—INV in tables 2A, 2B and 2C. These results corresponded to non-detection of the species of interest SOI, but were uninterpretable because the minimum detectable concentration Cmin_{SOI} was higher than the metagenomic threshold (SM). This result particularly differs from the results of microbiological culture, which generally produces negative results unless some device is used to individually validate the sensitivity of detection of a bacterial species in the tested sample. Validation with the metagenomic test allowed the risk of false negatives to be limited, this situation clearly being illustrated by the non-detection of E. cloacae in sample 27.

Comparison of the results of detection of pathogens of interest infecting the patients from whom the BAL and mini-BAL samples were taken, see table 3, clearly showed the advantage of using the control species described in this invention. Detection of pathogens above the clinical decision threshold, directly on the basis of the normalized number of reads assigned to the species of interest, produced almost 9 times more false positive results. Use of the control species allowed a significant improvement to the specificity of the metagenomic test and a better detection of infections, without loss of sensitivity.

TABLE 3

| True positive | | 10 |
|---|---|---|
| False positive | Unconfirmable | 14 |
| | Confirmed by MetaPhlAn and/or BLAST | 5 |
| | Negated by MetaPhlAn and/or BLAST | 0 |

| | |
|---|---|
| True negative | 586 |
| False negative | 0 |
| Positive predictive value | +34.5% |
| Negative predictive value | +100.0% |
| Sensitivity | +100.0% |
| Specificity | +96.9% |

A particular application of the invention to so-called shotgun sequences has been described. The invention is also applicable to targeted sequences, for example to so-called 16S sequences. In this case, prior to sequencing, a step of amplifying targeted genes was carried out in order to multiply the copies thereof in the sample. The reads used by the invention are then reads corresponding solely to the targeted genes.

The use of Bacillus subtilis as control species in a metagenomic analysis of BAL or mini-BAL samples has been described. As a variant, another control species may be used, provided that it meets all or some of the criteria described with reference to step 20. It may for example be a question of a species chosen from: Bacillus stearothermophilus, Synechocystis sp. PCC6803, Pelagibacter ubique, Methanocaldococcus jannaschii, Aeropyrum pernix, Kocuria rhizophila, Azospirillum lipoferum, Lactococcus lactis, Synechococcus sp. WH 7805, Schizosaccharomyces pombe, Pantoea stewartii, Phage T4, Pichia pastoris, and Armored DNA Quant™.

A plurality of control species taking the form of elements comprising nucleic acids comprised or encapsulated in membranes (bacterial membrane, capsid, etc.) have been described. This feature is used with respect to the function of validating conformity of the metagenomic analysis, and in particular to determine whether the process of extracting nucleic acids has worked as expected. Obviously, when a biological species is employed solely as calibrator, i.e. does not allow the function of validating conformity but solely the quantifying function to be performed, the calibrator may consist of free nucleic acids added to the sample or in a known quantity in the DNA extract.

Addition of control and calibration species at the same time, namely before the step of extracting the nucleic sequences, has been described. When two different biological species are used to perform, separately, the functions of validation of conformity and of quantification (calibrator), the calibrators may be added in a subsequent step, preferably after the step of lyzing the sample, when it is a question of naked nucleic acids, in order to avoid destruction of the latter.

The method according to the invention notably allows biological species of interest in a sample to be assayed. Preferably, in the context of a clinical application, the method according to the invention is completed by a step of determining a course of antibiotics depending on the species identified and assayed in the sample, and of administering the determined course of antibiotics to the patient.

The method allows assistance to be provided in diagnosis of a contamination of a sample by a species of interest, the latter possibly being a bacterium or a fungus. This allows a suitable treatment (antibiotic treatment in the case of a bacterium, antifungal treatment in the case of a yeast or of a fungus) to be defined, on the basis of the identity of the species of interest, but also on the basis of any signs of antimicrobial resistance detected in the genome.

More generally, depending on the targeted application, when the concentration of the biological species is higher than the decision threshold, this may be considered to be indicative of the occurrence of an anomaly. A suitable remedial course of action is decided upon, with a view to remedying the anomaly. For example, in the field of food processing, the species of interest may be a bacterium. When the concentration exceeds a certain threshold, the remedial course of action may be removal or destruction of food products intended to be sold, and/or cleaning of a production facility. The same applies when the application relates to sanitary inspection, for example sanitary inspection of a facility, for example part of a hospital, so as to prevent nosocomial infections. The acknowledged presence of an undesirable biological species leads to a remedial course of action such as cleaning or decontamination.

The invention will possibly be implemented in the health field, to assist with diagnosis, or, more generally, in the field of analysis of samples taken from the environment, or from industrial processes, for example in the food-processing industry, the pharmaceutical industry or the cosmetic industry. It may also be employed in sanitary inspection.

1. A method for detecting a biological species of interest (SOI) potentially present in an analysis sample, the biological species of interest having a known or partially known genome, the analysis sample comprising a mixture of various biological species, the method comprising:
a) extracting nucleic acids from the analysis sample;
b) sequencing nucleotide sequences extracted in a) the extracting;
c) on the basis of the result of the sequencing, performing;
(i) assigning the sequences resulting from b) the sequencing, based on a reference database of sequences;
(ii) determining a quantity of sequences assigned to the biological species of interest;
wherein the method further comprises, prior to b) the sequencing, adding a calibrator, the calibrator being a biological species added in a known concentration, to the analysis sample, the calibrator having a known genome, and wherein c) the performing on the basis of the result of the sequencing comprises
(iii) determining a quantity of sequences assigned to the calibrator,
d) on the basis of the quantities of sequences estimated in (ii) the determining of the quantity of sequences assigned to the biological species of interest and (iii) the determining of the quantity of sequences assigned to the calibrator, and of the concentration of the calibrator, estimating a concentration of the biological species of interest (SOI) in the sample.

2. The method of claim 1, wherein, in (ii) the determining of the quantity of sequences assigned to the biological species of interest and (iii) the determining of the quantity of sequences assigned to the calibrator, the quantities of sequences respectively assigned to the biological species of interest and to the calibrator are normalized by a reference quantity.

3. The method of claim 1, comprising taking into account a decision threshold, to which the concentration of the species of interest is compared.

4. The method of claim 1, wherein the sample comprising endogenous organisms, the calibrator has a genome different from that of the endogenous organisms.

5. The method of claim 1, wherein the calibrator is so that the size of its genome is comprised in a range of from 0.1 times to 10 times the size of the genome of the biological species of interest.

6. The method of claim 3, wherein the concentration of the calibrator is comprised in a range of from 0.001 times to 1000 times the decision threshold.

7. The method of claim 1, wherein d) the estimating of the concentration of the biological species of interest in the sample comprises:
determining a first ratio, between the quantities of sequences respectively assigned to the biological species of interest and to the calibrator;
determining a second ratio, between the respective genome sizes of the calibrator and of the biological species of interest;
taking into account the concentration of the calibrator added to the analysis sample.

8. The method of claim 7, wherein d) the estimating of the concentration of the biological species of interest in the sample comprises computing a product of the first ratio multiplied by the second ratio and by the concentration of the calibrator added to the analysis sample.

9. The method of claim 1, wherein d) the estimating of the concentration of the biological species of interest in the sample comprises:
determining a coverage for the biological species of interest and for the calibrator;
computing a ratio between the coverage determined for the biological species of interest and the coverage determined for the calibrator;
multiplying the ratio thus computed by the calibrator concentration added to the sample.

10. The method of claim 3, further comprising, following d) the estimating of the concentration of the biological species of interest in the sample, e) taking into account the decision threshold and comparing the concentration resulting from d) the estimating of the concentration of the biological species of interest in the sample with the decision threshold.

11. The method of claim 3, wherein the concentration of the calibrator is comprised in a range of from 0.01 to 100 times the decision threshold.

12. The method of claim 2, comprising taking into account a decision threshold, to which the concentration of the species of interest is compared.

13. The method of claim 2, w herein the sample comprising endogenous organisms, the calibrator has a genome different from that of the endogenous organisms.

14. The method of claim 3, wherein the sample comprising endogenous organisms, the calibrator has a genome different from that of the endogenous organisms.

15. The method of claim 12, wherein the sample comprising endogenous organisms, the calibrator has a genome different from that of the endogenous organisms.

16. The method of claim 2, wherein the calibrator is so that the size of its genome is comprised in a range of from 0.1 times to 10 times the size of the genome of the biological species of interest.

17. The method of claim 3, wherein the calibrator is so that the size of its genome is comprised in a range of from 0.1 times to 10 times the size of the genome of the biological species of interest.

18. The method of claim 4, wherein the calibrator is so that the size of its genome is comprised in a range of from 0.1 times to 10 times the size of the genome of the biological species of interest.

19. The method of claim 12, wherein the calibrator is so that the size of its genome is comprised in a range of from 0.1 times to 10 times the size of the genome of the biological species of interest.

20. The method of claim 13, wherein the calibrator is so that the size of its genome is comprised in a range of from 0.1 times to 10 times the size of the genome of the biological species of interest.
